(19) United States
(12) Patent Application Publication
(10) Pub. No.: US 2015/0192568 A1
(43) Pub. Date: Jul. 9, 2015

(54) METHOD AND APPARATUS FOR DETERMINING DEGREE OF STRATIFICATION AND/OR DIFFERENTIATION

(71) Applicant: Hitachi, Ltd., Tokyo (JP)

(72) Inventors: Keisuke Shibuya, Tokyo (JP); Ryoichi Haga, Tokyo (JP); Masaru Namba, Tokyo (JP); Ryota Nakajima, Tokyo (JP)

(73) Assignee: HITACHI, LTD., Tokyo (JP)

(21) Appl. No.: 14/419,185

(22) PCT Filed: Jul. 17, 2013
(86) PCT No.: PCT/JP2013/069336

§ 371 Date: Feb. 2, 2015

(30) Foreign Application Priority Data
Aug. 29, 2012 (JP) 2012-188823

(51) Int. Cl. G01N 33/50

(52) U.S. Cl. CPC G01N 33/5038; G01N 33/5091; G01N 2400/00

(57) ABSTRACT

A method for easily and non-invasively determining a degree of stratification and/or differentiation of cultured cells, and an apparatus for determining a degree of stratification and/or differentiation of cultured cells are provided. The invention relates to a method for determining a degree of stratification and/or differentiation of cultured cells, including: (a) a harvesting step of harvesting a culture solution of the cultured cells; (b) an analytical step of analyzing at least one type of a metabolite of the pentose phosphate pathway and/or the TCA cycle in the culture solution; and (c) a determination step of referring to a database of correlations between prospectively-obtained degrees of stratification and/or differentiation of the cultured cells and analytical results of metabolites, for analytical results obtained in the analytical step, to determine the degree of stratification and/or differentiation of the cultured cells.

### TECHNICAL FIELD

The present invention relates to a method for easily and non-invasively determining a degree of stratification and/or differentiation of cultured cells, and an apparatus for determining a degree of stratification and/or differentiation of cultured cells.

### BACKGROUND ART

In regenerative medicine, functions of tissues or organs, which malfunction due to diseases, or injuries, are recovered by using cells (stem cells) which are an origin of the skin, nerves, bones, blood vessels, etc. As to types of stem cells, mainly, there are (1) somatic stem cells, (2) ES cells and (3) iPS cells, and these stem cells have the characteristics described below.

### (1) Somatic Stem Cells

Somatic stem cells are immature cells which are included in a minute amount in the bone marrow, fats, the blood, etc. Somatic stem cells are present in the bone marrow, fats, etc. in the body, and differentiate into certain types of cells. Stem cells which become blood cells when they grow are called hematopoietic stem cells, stem cells which become bone cells, fat cells, etc. when they grow are called mesenchymal stem cells, and stem cells which become nerves in the brain are called neural stem cells. Since somatic stem cells are harvested from a patient, regenerative medicine using somatic stem cells has an advantage in which any rejection is not caused.

### (2) ES Cells

ES cells are produced from a part of a fertilized egg, and have a function to become various cells of the body. (3) iPS cells are produced by introducing genes into cells such as of the skin. iPS cells are considered to become various cells in the same manner as ES cells.

For example, in regenerative medicine, stem cells of undifferentiated corneal cells or oral cells, which have been harvested from the body, are proliferated and cultured, and cell sheets produced by properly stratifying the stem cells and/or inducing differentiation of the stem cells can be used for therapies.

For all the above-mentioned stem cells, their harvested amounts are small. Therefore, it is required that the harvested cells are multiplied to a sufficient amount and that induction of their differentiation into appropriate cells is carried out. Cell sheets can be produced by the following steps: (1) separation/purification of harvested stem cells; (2) culturing of the stem cells (increased to a required cell number); (3) induction of differentiation of stem cells into cells an intended tissue; (4) formulation/commercialization of differentiated cells into an appropriate form. These steps are all carried out under aseptic conditions, and there are often cases where a cell processing center is required in order to prevent contamination of other cells.

Furthermore, in the above-described steps, it is required to confirm whether the induction of differentiation is properly conducted, and to monitor degrees of the stratification and differentiation in order to determine appropriate timing of transplantation. As a method for determining degrees of the stratification and differentiation, techniques described below have conventionally been known.

### Staining Methods:

In hematoxylin/eosin staining (H&E staining), the basal layer, the stratum spinosum, the granular layer and the stratum corneum can be distinguished. In addition, localization of components of the basal lamina can be confirmed by using epidermal differentiation markers. However, when staining methods are used, it is required that a substance which serves as a marker is attached to cells. Therefore, there has been a problem in which the stained cells cannot be used as materials for therapies.

### Optical Methods:

Cited Literature 1 describes a method in which an optical measurement is used for easily and appropriately measuring a degree of stratification without requiring any steps of staining, transfer, etc. A confocal laser microscope is used to observe autofluorescence emitted from samples of harvested horny cell layers, and image data divided in the height direction of the horny cell layer are compared and analyzed to measure a multiple-layer exfoliation state of the horny cell layers.

Cited Literature 2 describes as follows: an enlarged image of a specimen of horny layer cells which has been captured under a condition of incident light is imported as a picture; pictures of three domains including a black-and-white image as a background part, a part of horny layer cells overlapping other horny layer cells and a part of horny layer cells not overlapping any other horny layer cells are extracted; and, by using, as indexes, results of a multivariate analysis on physical quantities of the pictures, evaluation of the horny layer cells such as an exfoliation state of the horny layer cells is carried out. According to cited Literature 2, by the method, shapes of horny layer cells, particularly an exfoliation state of horny layer cells, under staining or non-staining conditions can be promptly and accurately grasped to thereby evaluate skin characteristics.

However, when an optical method is used, it is difficult to distinguish the stratification with a general microscope. Additionally, although, in a method using a confocal microscope, the stratification can be distinguished by three-dimensional reconstruction of an image, there is a problem in which it is difficult to determine a degree of the differentiation based on the shapes, since cells are densely packed in a stratified state.

### Detection of Components of a Culture Solution:

Cited Literature 3 describes a method for determining a degree of stratification based on time variations of cell survival-associated components, specifically glucose, lactic acid and ammonia, as indexes. However, although a degree of stratification can be determined according to the method described in cited Literature 3, it is difficult to determine a degree of progression of differentiation after induction of differentiation. To determine appropriate timing of harvesting cells, which is not too early or late, after induction of their differentiation, a method which is able to analyze a degree of progression of the differentiation even after induction of differentiation is required.

Additionally, measurement of a certain protein specific to differentiated cells which are a component of a culture solution using ELISA (Enzyme-Linked ImmunoSorbent Assay) is useful. However, when such a method is used, a long analysis time is required. Also, expensive antibody proteins are required, and, therefore, there is a problem in which costs for the analysis are high (cited Literature 4).

Thus, there have been the above-described problems in using conventional methods of determining a degree of stratification or differentiation of cultured cells, for production processes of cells for medical purposes.

### CITATION LIST

### Patent Literature

PTL 1: JP-A-2009-247570

PTL 2: JP-A-2006-95218

PTL 3: JP-A-2004-215585

PTL 4: JP-A-2007-228873

### SUMMARY OF INVENTION

### Technical Problem

A problem to be solved by the invention is to provide, a method for easily and non-invasively determining a degrees of stratification and/or differentiation of cultured cells, and an apparatus for determining a degree of stratification and differentiation of cultured cells.

### Solution to Problem

In consideration of the above-described current situation, the present inventors, found that a degree of stratification and/or differentiation of cultured cells can easily and non-invasively be determined by measuring metabolite of the pentose phosphate pathway and/or a metabolite of the TCA cycle (citric acid cycle) which are metabolized by the cells.

That is, the invention encompasses the following inventions.
(1) A method for determining a degree of stratification and/or differentiation of cultured cells, including:
(a) a harvesting step of harvesting a culture solution of the cultured cells;
(b) an analytical step of analyzing at least one type of a metabolite of the pentose phosphate pathway and/or the TCA cycle in the culture solution; and
(c) a determination step of referring to a database of correlations between prospectively-obtained degrees of stratification and/or differentiation of the cultured cells and analytical results of metabolites, for analytical results obtained in the analytical step, to determine the degree of stratification and/or differentiation of the cultured cells.
(2) The method according to above (1), wherein the metabolite of the pentose phosphate pathway is at least one type selected from the group consisting of glucose-6-phosphate, 6-phosphoglucono-1,5-lactone, 6-phosphogluconic acid, ribulose-5-phosphate, xylulose-5-phosphate, ribose-5-phosphate, glyceraldehyde-3-phosphate, sedoheptulose-7-phosphate, erythrose-4-phosphate, and fructose-6-phosphate.
(3) The method according to above (1) or (2), wherein the metabolite of the TCA cycle is at least one type selected from the group consisting of acetyl CoA, citric acid, cis-aconitic acid, isocitric acid, oxalosuccinic acid, α-ketoglutarate, succinyl CoA, succinate, ubiquinone, fumarate, ubiquinol, L-malate and oxaloacetate.
(4) The method according to any one of above (1) to (3), wherein the database of correlations is based on correlations between the degrees of stratification and/or differentiation and production rates and/or consumption rates of the metabolites.
(5) The method according to any one of above (1) to (4), where the database of correlations is based on analysis results from an intracellular metabolic flux analysis.
(6) The method according to any one of above (1) to (5), wherein the metabolite analyzed in Step (b) is selected based on analysis results from an intracellular metabolic flux analysis.
(7) The method according to any one of above (1) to (6), wherein the analytical results of Step (b) are statistically compared with the database of correlations in Step (c).
(8) An apparatus for determining a degree of stratification and/or differentiation of cultured cells, including:

a harvesting means which harvests a culture solution of the cultured cells;

an analytical means which analyzes at least one type of a metabolite of the pentose phosphate pathway and/or the TCA cycle in the culture solution harvested by the harvesting means; and

a determination means which refers to a database of correlations between prospectively-obtained degrees of stratification and/or differentiation of the cultured cells and analytical results of metabolites, for analytical results obtained by the analytical means, to determine the degree of stratification and/or differentiation of the cultured cells.
(9) The apparatus according to above (8), wherein the analytical means is HPLC.

### Advantageous Effects of Invention

According to the method and the apparatus for determining a degree of stratification and/or differentiation of the invention, a degree of stratification and/or differentiation of cells can be measured easily and non-invasively with respect to the cells.

Any problems, elements and effects other than those described above will be clarified by the description of embodiments below.

### BRIEF DESCRIPTION OF DRAWINGS

FIG. 1 is a diagram showing a method for an intracellular metabolic flux analysis.

FIG. 2 is a diagram showing pathways of intracellular metabolic reactions in general animal cells.

FIG. 3 is a diagram showing a flow chart of determination of the stratification/differentiation.

FIG. 4 is a diagram showing one embodiment of the apparatus of the invention.

FIG. 5 is a diagram showing isotope ratios of intracellular intermediate metabolites in human oral cells.

FIG. 6 is a diagram showing isotope ratios of intracellular intermediate metabolites in human corneal cells.

FIG. 7 is a diagram showing transfer of carbon positions in carbon skeletons in intracellular metabolisms.

FIG. 8 is a diagram showing intracellular metabolic changes in differentiation/stratification using an intracellular metabolic flux analysis with respect to human oral cells.

FIG. 9 is a diagram showing intracellular metabolic changes in differentiation/stratification using an intracellular metabolic flux analysis with respect to human corneal cells.

### DESCRIPTION OF EMBODIMENTS

The invention is a method for determining a degree of stratification and/or differentiation of cultured cells, and is characterized by including:
(a) a harvesting step of harvesting a culture solution of the cultured cells;
(b) an analytical step of analyzing at least one type of a metabolite of the pentose phosphate pathway and/or the TCA cycle in the culture solution; and
(c) a determination step of referring to a database of correlations between prospectively-obtained degrees of stratification and/or differentiation of the cultured cells and analytical results of metabolites, for analytical results obtained in the analytical step, to determine the degree of stratification and/or differentiation of the cultured cells. The method for determining a degree of stratification and/or differentiation according to the invention (hereinafter, referred to as a “method of the invention”) includes above Steps (a), (b) and (c) to thereby easily and non-invasively determine a degree of stratification and/or differentiation of cultured cells.

In the invention, the “differentiation” means that individual cells structurally or functionally change. In the invention, the “stratification” means that cells overlap each other in several layers to form a thickened plane. The “stratification” may even include a case of being formed by differentiation of cells, and, in that case, the stratification and the differentiation simultaneously occur.

The method of the invention includes, as Step (a), a harvesting step of harvesting a culture solution of cultured cells.

The above cells are not particularly limited as long as they are cells which stratify or differentiate. However, adhesion-dependent cells (cells which adhere directly or indirectly to a culture surface and which then extend their adhesion area, followed by cell division; also called anchorage-dependent cells) are preferable. As the above cells, various cells harvested from warm-blooded animals (e.g. humans, mice, rats; guinea pigs, hamsters, chickens, rabbits, pigs, sheep, cows, horses, dogs, cats and monkeys), preferably from humans, are preferable. As the above cells, for example, keratinized cells (keratinocytes), epithelium cells, mucosal cells, endothelial cells, fibroblasts, oral cells and corneal cells can be mentioned. Among them, human oral cells and human corneal cells are preferable. These cells may be primary cells which are harvested directly from tissues or organs, or may be cells obtained through several passages of subculturing of them. In particular, stem cells such as somatic stem cells, ES cells and iPS cells are preferable. Furthermore, these cells may be any of embryonic cells which are undifferentiated cells, pluripotent stem cells such as mesenchymal stem cells having multipotency, unipotent stem cells such as endothelial progenitor cells having unipotency, and cells completing differentiation. In addition, the cells may be those obtained by culturing one type of cells or by coculturing two or more types of cells.

As examples of a harvesting means which carries out Step (a), harvesting of a culture solution from a culture container by hand work inside a safety cabinet, and a device which carries out the sampling through a flow channel aseptically connected to a sampling port provided to the culture container with a pump or by force-feeding can be mentioned. In terms of secure asepsis, the device or the like which carries out the sampling through a flow channel aseptically connected to a sampling port provided to the culture container with a pump or by force-feeding is preferably used.

The method of the invention includes, as Step (b), an analytical step of analyzing at least one type of a metabolite of the pentose phosphate pathway and/or the TCA cycle in a culture solution.

In stratification of cells and differentiation of cells, changes occur in intracellular metabolisms depending on a state of cells. It is preferable that changes in intracellular metabolisms in each culture sate are observed, thereby selecting, as an index, a metabolite which undergoes a significant change in the metabolite concentration accompanying changes in a state of cells, because a degree of stratification/differentiation of the cells can more accurately be determined. Furthermore, in general, there are many impurities in a culture solution, and inclusion of significant measurement errors is possible. Therefore, determination accuracy is preferably increased by using a plurality of metabolites as indexes. For selection of metabolites which serve as indexes, an intracellular metabolic flux analysis is preferably used, because, according to the analysis, it is possible to analyze pathways of intracellular metabolic reactions at once, and candidate components which possibly serves as indexes can be found out at once. For example, an metabolic flux analysis can be carried out to each of (1) a growth phase of undifferentiated cells, (2) differentiation-induced early phase, (3) differentiation-induced middle phase (cells are generally used for transplantation at this point) and (4) a differentiation-induced late phase, a metabolite-consumption rate or production rate of each metabolite in metabolic pathways is obtained, and metabolites which undergo changes in production or consumption rates in each of cellular states (1) to (4) can be used as indexes. In the analytical step, a concentration or the like in a culture solution of a metabolite selected as an index is analyzed.

As examples of an analytical means which carries out Step (b), a biosensor using an immobilized-enzyme method, mass spectrometry such as the MALDI method, and liquid chromatography can be mentioned. HPLC or the like is preferably used because such a method makes it possible to simultaneously measure multiple components in the culture solution.

As examples of metabolites of the above-mentioned pentose phosphate pathway, glucose-6-phosphate, 6-phosphoglucono-1,5-lactone, 6-phosphogluconic acid, ribulose-5-phosphate, xylulose-5-phosphate, ribose-5-phosphate, glyceraldehyde-3-phosphate, sedoheptulose-7-phosphate, erythrose-4-phosphate, and fructose-6-phosphate can be mentioned. These can be used singularly, or two or more types can be combined. Among them, glucose-6-phosphate, ribulose-5-phosphate, xylulose-5-phosphate, ribose-5-phosphate, glyceraldehyde-3-phosphate, sedoheptulose-7-phosphate, fructose-6-phosphate and erythrose-4-phosphate are preferable. Glucose-6-phosphate, glyceraldehyde-3-phosphate, sedoheptulose-7-phosphate, fructose-6-phosphate and erythrose-4-phosphate are particularly preferable.

As examples of metabolites of the above-mentioned TCA cycle, acetyl CoA, citric acid, cis-aconitic acid, isocitric acid, oxalosuccinic acid, α-ketoglutarate, succinyl CoA, succinate, ubiquinone, fumarate, ubiquinol, L-malate and oxaloacetate can be mentioned. These can be used singularly, or two or more types can be combined. Among them, oxaloacetate, acetyl CoA, citric acid, α-ketoglutarate, succinyl CoA, succinate, fumarate and L-malate are preferable. Citric acid, α-ketoglutarate, succinyl CoA, succinate and fumarate are particularly preferable.

The method of the invention includes, as Step (c), determination step of referring to a database of correlations between prospectively-obtained degrees of stratification and/or differentiation of the cultured cells and analytical results of metabolites, for analytical results obtained in the analytical step, to determine the degree of stratification and/or differentiation of the cultured cells

The database of correlations is preferably based on correlations between the degree of stratification and/or differentiation and production rates and/or consumption rates of the metabolites. For example, the production rates and/or the consumption rates can be determined based on isotope ratios of respective metabolites in the culture solution of cells obtained by culturing them in a culture medium including isotope-labeled glucose.

The database of correlations is particularly preferably based on analysis results according to an intracellular metabolic flux analysis, because progression of stratification and differentiation as well as production rates and/or consumption rates of intracellular intermediate metabolites can be examined in more detail. Hereinafter, a principle of the intracellular metabolic flux analysis will be described below.

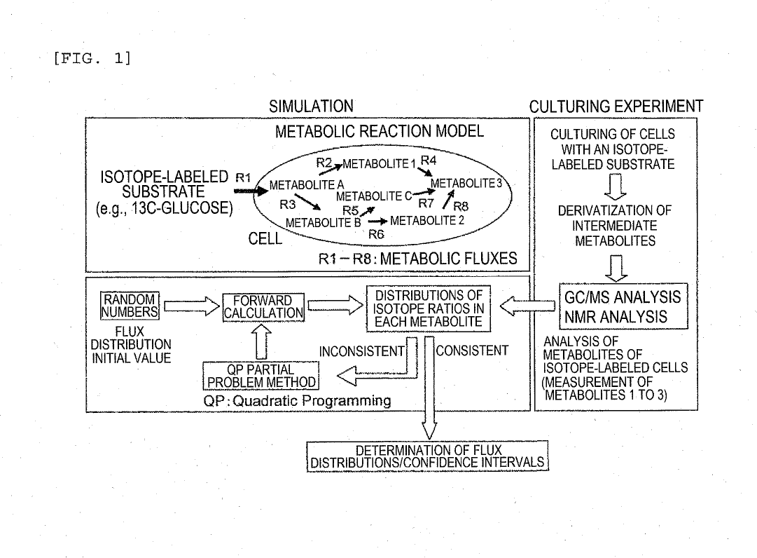

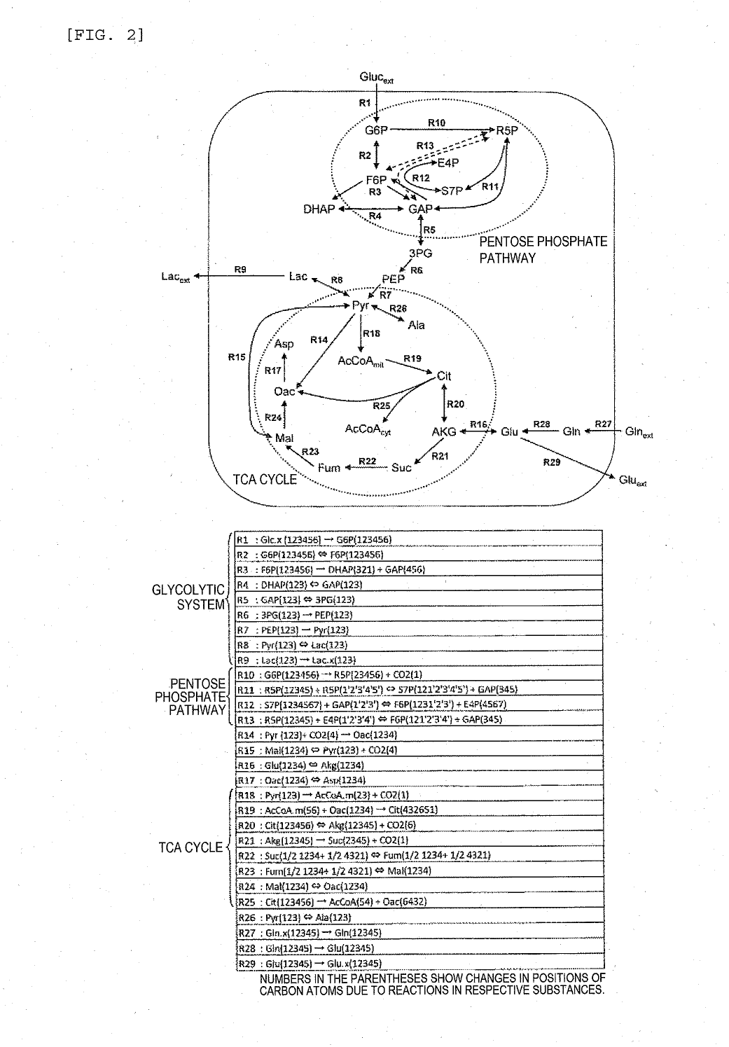

The intracellular metabolic analysis includes simulation and a culturing experiment (see FIG. 1). In the simulation, it is required to suppose a certain intracellular metabolic pathway of cells which is an analysis target. In the method of the invention, the model of metabolic pathways shown in FIG. 2 which is generally utilized in animal cells can be used. However, the model of metabolic pathways can also be modified depending on cells used therein or accuracies. A concept of a method for deducing metabolic fluxes is shown in FIG. 1. In the simulation, at first, random values (R1 to R29 in FIG. 2) of metabolic fluxes are given, and ratios of the number of carbon isotopes included in each of intracellular metabolic substances in a steady state is calculated based on the metabolic pathways in FIG. 2. A comparison between the calculated values and the ratios of the number of carbon isotopes of the intracellular metabolic substances measured in the experiment is made, and, when there is a statistically significant difference between them (they are different), the values for the metabolic fluxes according to the simulation is modified such that mean square errors of values of carbon isotope number ratios in the metabolic substances according to the simulation and values of carbon isotope number ratios in the metabolic substances according to the experiment become minimum, and a carbon isotope ratios are calculated. Then, until any statistically significant difference is not present, the above-described manipulation of comparison with the ratios of carbon isotope numbers in metabolic substances according to the experiment is repeated. Generally, the metabolic fluxes can be deduced by repeating the calculation 2 or 3 times. However, when a statistically significant difference is caused even when the calculation is repeated over and over again, it is deemed that the metabolic pathway model is wrong or that experimental data are not appropriately obtained. Additionally, in the method of the invention, confidence intervals for estimated values can also be obtained based on a statistical approach. For calculation of confidence intervals for estimated values, Maciek R. et al., Metabolic Engineering 8 (2006) 324-337, “Determination of confidence intervals of metabolic fluxes estimated from stable isotope measurements” can be, referred to. At first, estimated metabolic fluxes are obtained, and, focusing on one flux (for example, R3) of the estimated metabolic fluxes (R1 to R29), the value for the metabolic flux is increased by small increments. Then, when a statistically significant difference is present in comparison with an experimental value, the value is considered as an upper limit for the metabolic flux. For the lower limit, the estimated flux value is decreased by small increments, and, when a statistically significant difference is present, the value is considered as the lower limit. By successively carrying out the manipulations to other metabolic fluxes, confidence intervals can be obtained for all metabolic fluxes.

In addition, meanings of abbreviations used in the present description are listed below.
AcCoA: Acetyl-CoA (Acetyl coenzyme A)
AKG: α-Ketoglutarate
Ala: Alanine
Asp: Aspartic acid
Cit: Citric acid
DHAP: Dihydroxyacetone phosphate
E4P: Erythrose-4-phosphate
Fum: Fumarate
F6P: Fructose-6-phosphate
GAP: Glyceraldehyde-3-phosphate
Gln: Glutamine
Glnext: Extracellular Glutamine
Gluc: Glucose
Glucext: Extracellular Glucose
Glu: Glutamic acid
Gly: Glycine
G6P: Glucose-6-phosphate
Lac: Lactic acid
Lacext: Extracellular Lactic acid
Mal: Malate
Oac: Oxaloacetate
PEP: Phosphoenolpyruvic acid
Pyr: Pyruvate
R5P: Ribose-5-phosphate
Ser: Serine
Suc: Succinate
SucCoA: Succinyl-CoA
S7P: Sedoheptulose-7-phosphate
Thr: Threonine
3PG: 3-phosphoglycerate

In the above-described simulation, observation parameters (isotope carbon number ratios of respective metabolic substances inside cells and extracellular metabolic fluxes) are required. However, in general, required observation parameters are different depending on target cells, a culture medium used therein, etc. In the method of the invention, 13 types of Pyr, Lac, Ala, Gly, Suc, Fum, Ser, AKG, Mal, Asp, Glu, Gln and Cit are used as measurement parameters, and, for these values, values obtained in the measurement of the isotope ratios can be used.

As examples of a determination means which carries out Step (c), a means which compares concentrations of intracellular metabolites with a database, a means which compares concentrations of metabolites secreted into the culture solution with a database, and a means which compares consumption rates of nutrients consumed in cells with a database based on changes of concentrations of nutrients in the culture solution can be mentioned. In particular, in terms of enhanced determination accuracy, a determination means or the like which calculates a metabolic rate per cell and which compares the calculated metabolic rate with a database is preferably used.

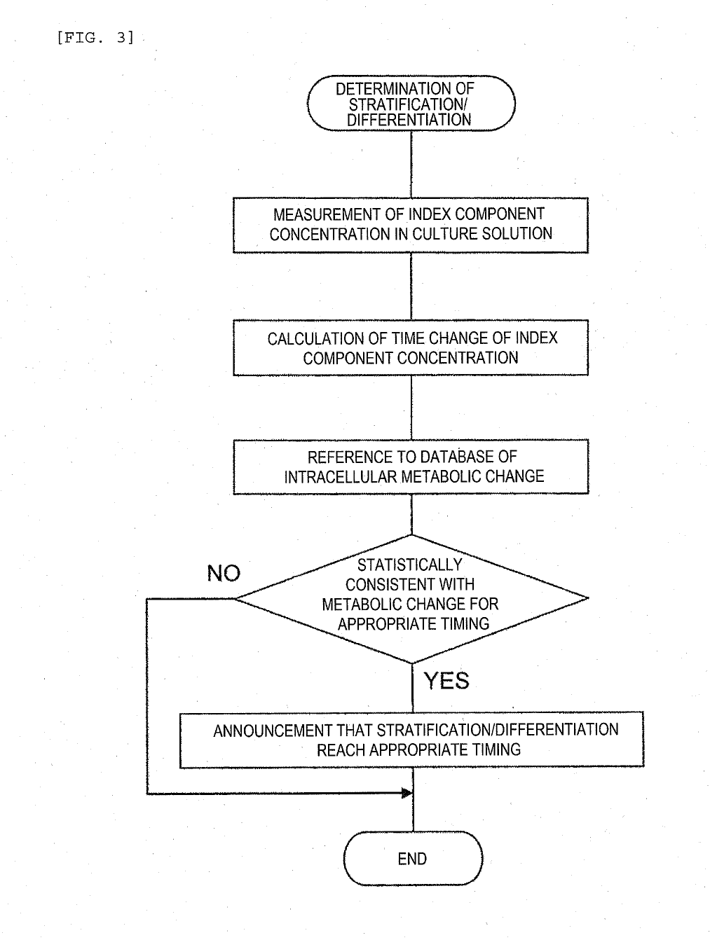

As an example which more highly accurately carries out the method of the invention, a method as shown in FIG. 3 can be mentioned. As shown in FIG. 3, since analytical results of Step (b) are statistically compared with the database of correlations in the method, the determination step of Step (c) can more highly accurately be carried out. The method shown in FIG. 3 will specifically be described below.

In advance, degrees of stratification and/or differentiation and time-course changes of index substances are recorded. The time-course changes of index substances may be time-course changes of metabolic fluxes, and, in that case, metabolic rates of index substances can be estimated from metabolic fluxes corresponding to metabolism of index substances. A culture solution of target cultured cells is harvested, and concentrations of index components in the culture solution are measured. The concentrations are measured over time, and variations are calculated. The variations are compared with a database of correlations which has been obtained in advance, and, when they are statistically consistent with each other, it can be determined that the cultured cells are in a cellular state referred to by the consistent database.

The invention further relates to an apparatus (hereinafter, referred to as an “apparatus of the present invention”) for determining a degree of stratification and/or differentiation of cultured cells, including:

a harvesting means which harvests a culture solution of the cultured cells;

an analytical means which analyzes at least one type of a metabolite of the pentose phosphate pathway and/or the TCA cycle in the culture solution harvested by the harvesting means; and

a determination means which refers to a database of correlations between prospectively-obtained degrees of stratification and/or differentiation of the cultured cells and analytical results of metabolites, for analytical results obtained by the analytical means, to determine the degree of stratification and/or differentiation of the cultured cells. The apparatus of the invention is suitable for carrying out the method of the invention. In other words, according to the apparatus of the invention, a degree of stratification and/or differentiation of the cells can be measured easily and non-invasively with respect to cells.

In the apparatus of the invention, it is preferable that a pathway from harvesting to analysis of the culture solution is formed by a closed system for the purpose of prevention of contamination from the outside.

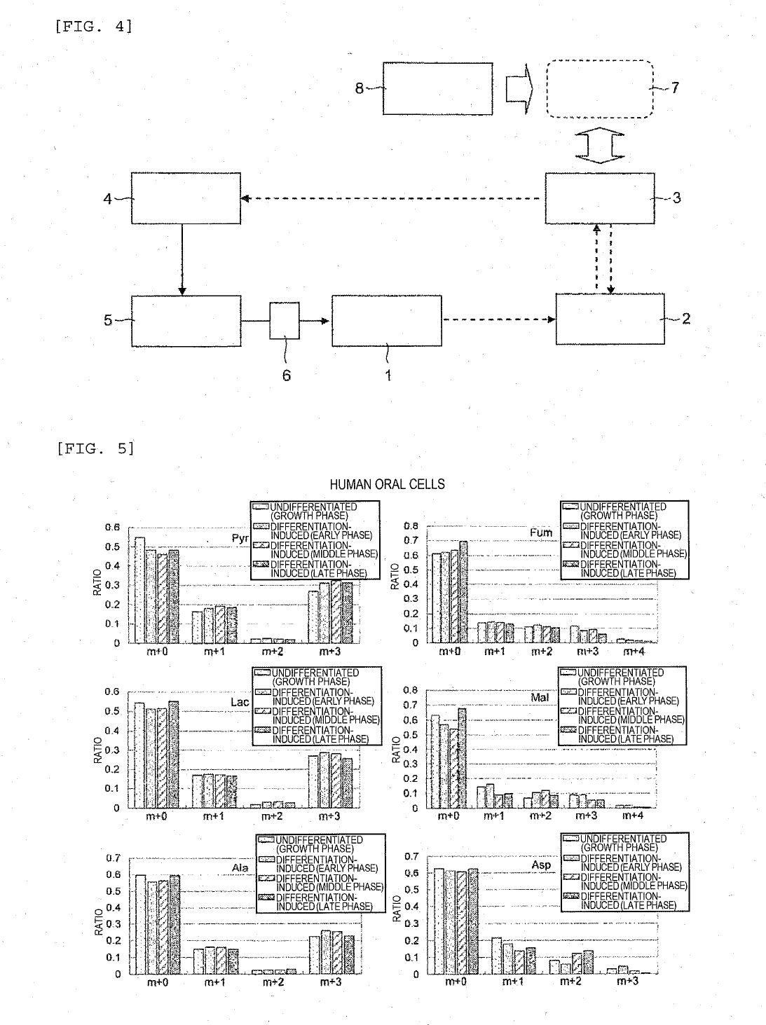

One embodiment of the apparatus of the invention is shown in FIG. 4. In the embodiment, a culturing device, a harvesting device, an analyzing device, an analysis device, a recording device, a database of correlations, and a controlling device are provided. By using the apparatus according to the embodiment, the stratification/differentiation of cultured cells can be determined, for example, in the following way. Culturing for formation of cell sheets is carried out with the automatic culturing device, a portion of the culture solution is harvested with the harvesting device during replacement of the culture medium. The harvested culture solution is analyzed in the analyzing device, calculations are carried out, according to necessity, with respect to the obtained results with the analysis device, and results are recorded in the recording device. By crosschecking the measured results to the database of correlations, a current cellular state is determined.

### EXAMPLES

1. Determination of Indexes for Determination of a Degree of Stratification and/or Differentiation

Human oral cells or human corneal cells were used to carry out culturing for formation of cell sheets. Cell samples of (1) an undifferentiated growth phase, (2) a differentiation-induced early phase, (3) a differentiation-induced middle phase and (4) a differentiation-induced late phase were each prepared, and an intracellular metabolic flux analysis was carried out.

### 1.1 Culturing for Cell Sheet Formation

### (1) Cells and Media

Human oral cells or human corneal cells were used for the experiment. For proliferation culturing, a medium obtained by mixing the Dulbecco's Modified Eagle Medium (DMEM) and the F12 medium at 1:1, followed by addition of 10% bovine serum, was used. However, glucose was prepared such that a proportion of native glucose, a proportion of 1-13C glucose in which position 1 of the carbon skeleton was labeled with the isotope, and a proportion of U-13C glucose in which 6 carbons of the carbon skeleton were all labeled with the isotope were 50%, 25% and 25%, respectively. On the other hand, the KCM media (keratinocyte culture media) were used for the stratification and differentiation induction. In the same manner as proliferation culturing, glucose was prepared such that proportions of native glucose, 1-13C glucose and U-13C glucose were 50%, 25% and 25%, respectively.

### (2) Culturing Method

Human oral cell or human corneal cells were seeded to 6-well plates having a temperature-responsive film, the cells were cultured in the above-described DMEM/F12 media including the isotopic glucose in an incubator (37° C., 5% CO₂, >95% humidity), and replacement of culture media was carried out every 3 or 4 days. When the cells were proliferated to a confluent state, the culture media were replaced with the culture media for differentiation induction (KCM media) to differentiate them. After replacement with the culture media for differentiation induction, replacement of the culture media was carried out with the KCM media every 3 or 4 days, thereby stratifying the cells and thus forming cell sheets.

### 1.2 Measurement of Isotope Ratios of Intracellular Intermediate Metabolites

### (1) Analyzing Method

The 6-well plates were taken out of the incubator, and the isotope-labeled cells were washed with a PBS solution once, followed by removal of the washing solution. Then, 200 μL of methanol which had been cooled to −20° C. was added to each well, and was spread all over the well. The plates were transferred on ice, and 600 μL of distilled water was added to each well. The surface of the well was scraped with an end of a tip for Pipetman to strip cells, and the cells were transferred into a microtube which had been cooled on ice. The cells were sonicated for 1 minute, and then, 800 μL of chloroform was further added thereto. The mixture was mixed with a vortex mixer at 4° C. for 30 minutes. The mixture was centrifuged (11, 500 rpm, 4° C., 30 minutes) with a centrifugal machine (“Microfuge R”; Beckman Coulter, Inc.), and an upper layer of divided two layers was transferred to another microtube. The sample was then dried by evaporation overnight.

30 μL of 2% methoxyamine hydrochloride (O-methylhydroxylamine hydrochloride) (Pierce Inc.) was added to each dried sample. The sample was briefly vortexed, and the solution was collected to the middle and bottom portions with a table-top centrifuge. Then, the mixture was reacted on a heat block at 37° C. for 2 hours. 45 μL of a MTBSTFA (N-methyl-N-t-butyldimethylsilyltrifluoroacetamide) +1% TBDMCS (t-butyldimethylsilyl) solution (Pierce Inc.) was added to each mixture. Then, the mixture was briefly vortexed, and the solution was collected to the middle and bottom portions with a table-top centrifuge, and then, was reacted on a heat block at 55° C. for 1 hour. The reaction solution was transferred to a container for GC/MS analysis, and was stored at an ordinary temperature until the analysis.

For the GC/MS analysis, “Agilent 7890A” (Agilent Technologies), and Column Type “30 m DB-35MS capillary column” were used. The analysis was carried out under measuring conditions where the column temperature gradient was 3.5° C./min with a temperature control of 100° C. to 300° C., the temperature of the injection port was 270° C., a carrier gas was helium gas, and the flow rate was 1 mL/min.

### (2) Analytical Results

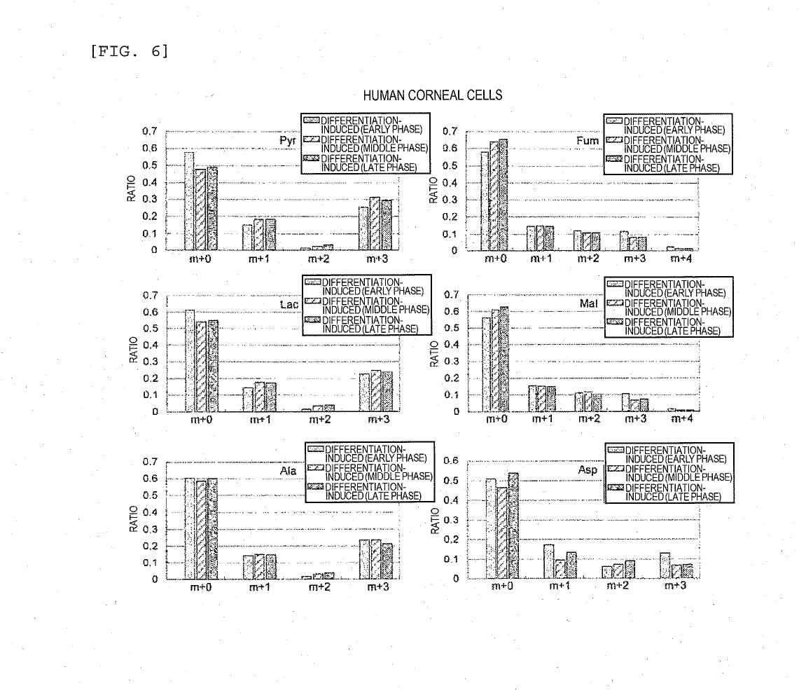

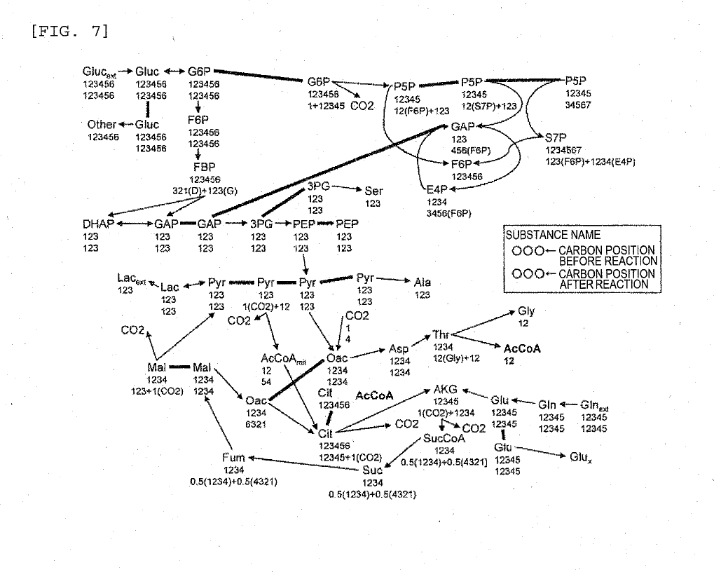

Analytical results for human oral cells are shown in FIG. 5, and analytical results for human corneal cells are shown in FIG. 6. In addition, X of the description of (m+X) in FIG. 5 and FIG. 6 shows a number of isotope-labeled carbons in an intracellular metabolite.

With regard to pyruvate (Pyr), the proportion of isotope-unlabeled pyruvate (m+0) was the largest in the undifferentiated growth phase (1), and was decreased during the differentiation induction (2). However, the proportion of isotope-unlabeled pyruvate (m+0) was slightly increased in the differentiation-induced late phase (4).

A map which represents transfer of positions of carbon atoms in the carbon skeleton of each intracellular metabolite is shown in FIG. 7. Isotopic glucose was intracellularly metabolized to pyruvate. In this process, the carbon atom at position 1 is released as carbon dioxide in a path (G6P [symbol] P5P) which fluxes in the pentose phosphate pathway (see FIG. 7 and FIG. 2). To the contrary, a carbon atom at position 1 are not released in any other paths. Since 25% of 1-13C glucose was combined in this experiment, when the flux to the pentose phosphate pathway is large, metabolites derived from 1-13C glucose lose the isotope label, and the metabolism to pyruvate is influenced. As a result, when the flux to the pentose phosphate pathway becomes large, the proportion of isotope-unlabeled pyruvate (m+0) is increased. In view of this consideration, it was understood that the activity of the pentose phosphate pathway is decreased in the undifferentiated growth phase (1) to the differentiation-induced middle phase (3), and that the activity of the pentose phosphate pathway is slightly recovered in the differentiation-induced late phase (4). This shows that evaluation of an increase or decrease of a metabolite in the pentose phosphate pathway makes it possible to determine a degree of progression of the stratification/differentiation.

With regard to fumarate, it was shown that the proportion of isotope-unlabeled fumarate (m+0) successively increased from undifferentiated growth phase (1) to the differentiation-induced late phase (4). This shows that there are paths, using unlabeled glutamine as a substrate, which flux into the TCA cycle, and that the paths (AKG [symbol] Glu, AKG [symbol] Suc, and Suc [symbol] Fum) are increased with the stratification/differentiation.

On the other hand, with regard to maleic acid (Mal), the proportion of isotope-unlabeled maleic acid (m+0) decreased from the undifferentiated growth phase (1) to the differentiation-induced middle phase (3), and then, increased in the differentiation-induced late phase (4). This shows that fluxes of Pyr [symbol] Oac, Oac [symbol] Mal and Pyr [symbol] Mal, to which a larger amount of the isotope-labeled substance flows, than to the flux of Mal [symbol] Fum, are increased in the undifferentiated growth phase (1) to the differentiation-induced middle phase (3), and that the fluxes of the metabolic pathway are decreased in the differentiation-induced middle phase (3) to the differentiation-induced late phase (4).

This shows that evaluation of an increase or decrease of a metabolite in the TCA cycle makes it possible to determine a degree of progression of the stratification/differentiation.

As seen from FIG. 5 and FIG. 6, although human oral cells and human corneal cells are of different origins, tendencies of their isotope ratios in the stratification/differentiation are similar to one another. Therefore, it is considered that tendencies of changes in metabolite production or metabolite consumption rates accompanying the stratification/differentiation are the same between them.

### 1.3 Analysis on Intracellular Metabolism

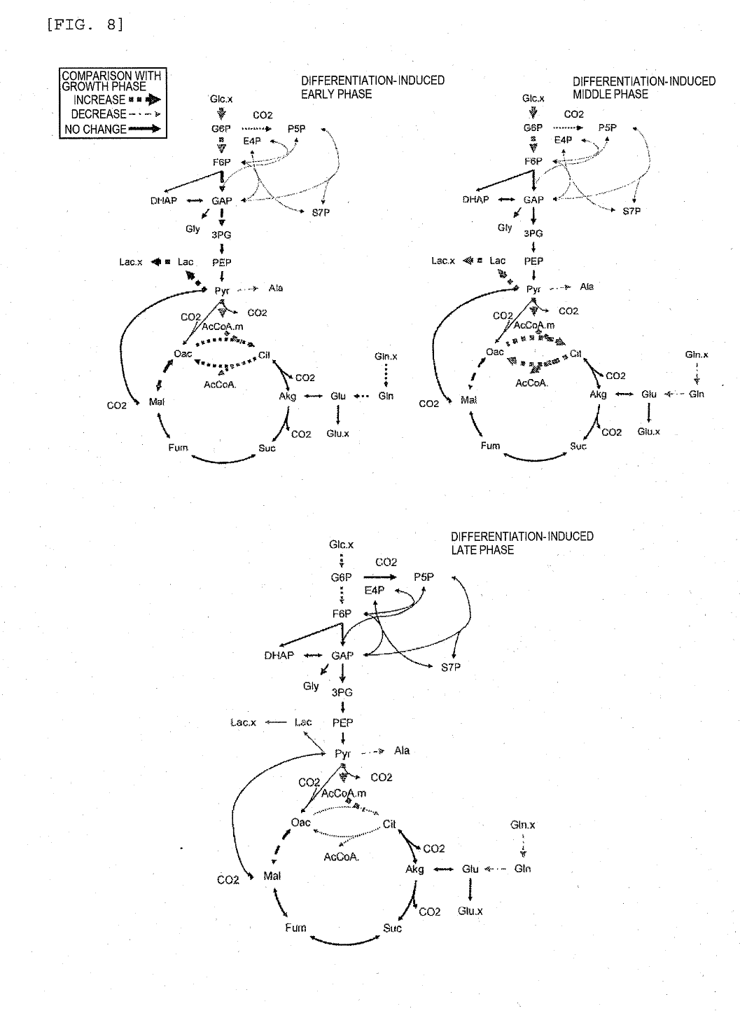

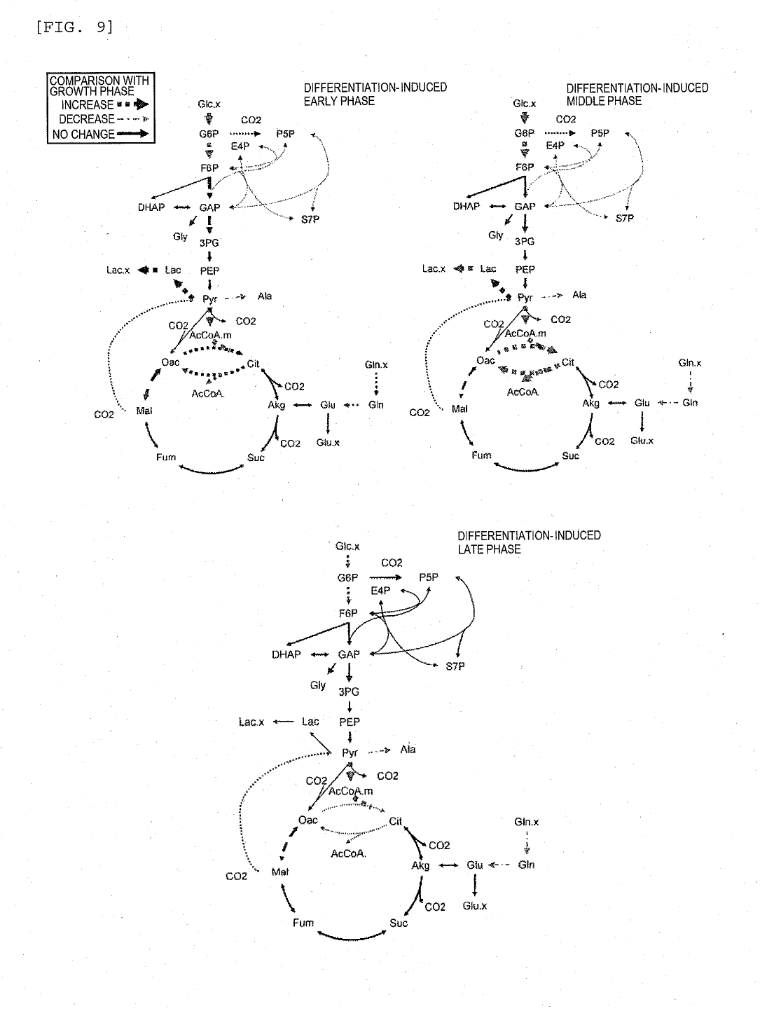

By using analytical results of 1.2 (2), the simulation shown in FIG. 1 was carried out. The analysis results for human oral cells are shown in FIG. 8. Entire fluxes of the pentose phosphate pathway were decreased when the formation of derivatives started in the differentiation-induced early phase. On the other hand, fluxes of the whole TCA cycle were increased, and, in particular, fluxes of Mal [symbol] Oac and Oac [symbol] Cit were increased. The above increasing tendencies were enhanced in the differentiation-induced middle phase. In the differentiation-induced late phase, fluxes of Mal [symbol] Oac and Oac [symbol] Cit in the TCA cycle were decreased, and other fluxes of the TCA cycle became equal to fluxes in the undifferentiated state. The tendencies of increase/decrease in metabolic fluxes for human corneal cells were consistent with those of the human oral cells.

Analysis results for human corneal cells are shown in FIG. 9. The same tendencies as human oral cells were exhibited except that Pyr [symbol] Mal became small.

Based on the results, it was revealed that increases or decreases in all metabolic components of the pentose phosphate pathway and the TCA cycle can be used as indexes for determination of the stratification/differentiation.

Based on the analytical results for the case of human oral cells shown in FIG. 8 and the analytical results for the case of human corneal cells shown in FIG. 9, it was revealed that determination of the stratification/differentiation is carried out favorably by using, as indexes, glucose-6-phosphate, glyceraldehyde-3-phosphate, sedoheptulose-7-phosphate, fructose-6-phosphate, erythrose-4-phosphate, citric acid, α-ketoglutarate, succinyl CoA, succinate and fumarate.

### 2. Determination of Degrees of Stratification and Differentiation

A target culture solution in culturing for cell sheet formation was sampled, and index components in the culture solution are analyzed. This is measured over time, and variations are calculated with an analysis device. By comparing the variations calculated in the analysis device with the database obtained in advance, it is determined which cellular state the current culture corresponds to.

In addition, the invention is not considered to be limited to the above-described examples, and includes various variation examples. For example, the above examples are described in detail in order to clearly describe the invention, and is not necessarily limited to those including all the described elements. Moreover, a part of elements of one example can be replaced with elements of another example, and also, to elements of one example can be added elements of another example. Furthermore, for apart of elements of each example, addition/deletion/replacement of other elements are possible.

### INDUSTRIAL APPLICABILITY

The method and the apparatus for determining degrees of stratification and differentiation according to the invention can be used for quality control in a culturing step or for confirmation of appropriate timing for transplantation, particularly in relation to cells used for regenerative medicine. For example, the method and the apparatus according to the invention can be used for cell monitoring used when culturing cells used for regenerative medicine.

### REFERENCE SIGNS LIST

1: an analyzing device
2: a recording device
3: an analysis device
4: a controlling device
5: a culturing device
6: a harvesting device
7: a database of correlations
8: culturing experiments and metabolism analysis

1. A method for determining a degree of stratification and/or differentiation of cultured cells, comprising:
(a) a harvesting step of harvesting a culture solution of the cultured cells;
(b) an analytical step of analyzing at least one type of a metabolite of the pentose phosphate pathway and/or the TCA cycle in the culture solution; and
(c) a determination step of referring to a database of correlations between prospectively-obtained degrees of stratification and/or differentiation of the cultured cells and analytical results of metabolites, for analytical results obtained in the analytical step, to determine the degree of stratification and/or differentiation of the cultured cells.

2. The method according to claim 1, wherein the metabolite of the pentose phosphate pathway is at least one type selected from the group consisting of glucose-6-phosphate, 6-phosphoglucono-1,5-lactone, 6-phosphogluconic acid, ribulose-5-phosphate, xylulose-5-phosphate, ribose-5-phosphate, glyceraldehyde-3-phosphate, sedoheptulose-7-phosphate, erythrose-4-phosphate, and fructose-6-phosphate.

3. The method according to claim 1, wherein the metabolite of the TCA cycle is at least one type selected from the group consisting of acetyl CoA, citric acid, cis-aconitic acid, isocitric acid, oxalosuccinic acid, α-ketoglutarate, succinyl CoA, succinate, ubiquinone, fumarate, ubiquinol, L-malate and oxaloacetate.

4. The method according to claim 1 wherein the database of correlations is based on correlations between the degrees of stratification and/or differentiation and production rates and/or consumption rates of the metabolites.

5. The method according to claim 1 where the database of correlations is based on analysis results from an intracellular metabolic flux analysis.

6. The method according to claim 1 wherein the metabolite analyzed in Step (b) is selected based on analysis results from an intracellular metabolic flux analysis.

7. The method according to claim 1 wherein the analytical results of Step (b) are statistically compared with the database of correlations in Step (c).

8. An apparatus for determining a degree of stratification and/or differentiation of cultured cells, comprising:
a harvesting means which harvests a culture solution of the cultured cells;
an analytical means which analyzes at least one type of a metabolite of the pentose phosphate pathway and/or the TCA cycle in the culture solution harvested by the harvesting means; and
a determination means which refers to a database of correlations between prospectively-obtained degrees of stratification and/or differentiation of the cultured cells and analytical results of metabolites, for analytical results obtained by the analytical means, to determine the degree of stratification and/or differentiation of the cultured cells.

9. The apparatus according to claim 8, wherein the analytical means is HPLC.
